(19) United States
(12) Patent Application Publication
(10) Pub. No.: US 2022/0096563 A1
(43) Pub. Date: Mar. 31, 2022

(54) COMPOSITION FOR TREATING INFLAMMATORY DISEASE INDUCED BY HYPERIMMUNE RESPONSE

(71) Applicants: KOLON LIFE SCIENCE, INC., Seoul (KR); KOLON TISSUEGENE, INC., Rockville, MD (US)

(72) Inventors: Heonsik CHOI, Seoul (KR); Sujeong KIM, Seoul (KR); Hyeonyoul LEE, Gyeonggi-do (KR); Jinwon SEO, Seoul (KR); Yunsin LEE, Seoul (KR); Heungdeok KIM, Seoul (KR); Kiwon PARK, Seoul (KR)

(21) Appl. No.: 17/543,911

(22) Filed: Dec. 7, 2021

Related U.S. Application Data
(63) Continuation-in-part of application No. 15/547,566, filed on Jul. 31, 2017, now Pat. No. 11,191,787.
(60) Provisional application No. 62/112,718, filed on Feb. 6, 2015.

(51) Int. Cl. A61K 35/32; C12N 5/077; C07K 14/495; A61P 29/00

(52) U.S. Cl. CPC A61K 35/32; C12N 5/0655; A61K 38/00; A61P 29/00; C07K 14/495

(57) ABSTRACT

A composition according to an embodiment of the present application includes at least one of a chondrocyte and a cell capable of differentiating into a chondrocyte, and a cell into which a gene encoding TGF-β1 is introduced and derived from human embryonic kidney 293 (HEK-293) cell. The composition may be used for treating an inflammatory disease induced by a hyperimmune response.

### CROSS-REFERENCE TO RELATED APPLICATION AND CLAIM OF PRIORITY

This application is a Continuation-In-Part of co-pending application Ser. No. 15/547,566, filed on Jul. 31, 2017, which is a National Stage entry under 35 U.S.C. § 371 of International Application No. PCT/KR2016/001310 filed on Feb. 5, 2016, which claims priority to the benefit of U.S. Provisional Patent Application No. 62/112,718 filed on Feb. 6, 2015, the entire contents of which are incorporated herein by reference.

### BACKGROUND

### 1. Technical Field

The present invention relates to a cell therapeutic agent, more specifically, to a cell therapeutic agent for treating an inflammatory disease induced by a hyperimmune response

### 2. Description of the Related Art

Degenerative arthritis is a disease caused by gradual loss of cartilage which protects the joints due to aging, and it is apt to occur in the knee joint which supports a significant portion of the body weight.

In contrast, rheumatoid arthritis of an autoimmune disease is a disease that our body's immune cells attack the joints to cause inflammation and inflammation of the joints continuously infiltrates into the articular cartilage and bone tissue, resulting in tissue erosion. Rheumatoid arthritis often occurs in fingers and wrists, and it is especially characterized by protruded middle nodes of the fingers. Unlike degenerative arthritis, which occurs mainly at the age of fifties or elder, rheumatoid arthritis often occurs at young ages.

The causes, symptoms, sites of onset, and ages of onset of degenerative arthritis and rheumatoid arthritis are compared to each other as follows (Table 1).

TABLE 1

| | Degenerative arthritis | Rheumatoid Arthritis |
|---|---|---|
| Cause | Aging, overweight, | Immune dysfunction |
| | repetition of severe | |
| | motion | |
| Symptom | Unnatural walking, Pain | Pain and flash at the joint |
| | after movement | site, Pain after break |
| Region | Large joints such as | Small joints such as |
| | knees, hips, hip joints | fingers, wrists, and toe |
| | | fingers |
| Age | Elderly people after 50s | All ages |

It has been reported that when rheumatoid arthritis occurs, the production of cytokines increases in joint macrophages or fibroblasts and the production of IFN-γ by Th1 cells and IL-17 cytokine by Th17 cells increases. Although cytokines produced in Th1 and Th17 cells exacerbate the arthritic symptoms, cytokines produced by Th2 cells such as IL-4 and IL-10 are known to prevent or treat arthritis. It has been reported that when a viral vector including IL-4 or IL-10 gene of a Th2 type cytokine is injected into a joint of an arthritis-induced mouse, the therapeutic effect is exerted not only at the injected leg but also at the other legs (S H Kim et al., J. Immunol., 166: 3499-3505 (2001)).

Small molecules that have been developed so far as therapeutic agents or reducing agents for rheumatoid arthritis include methotrexate, azathioprine, cyclophosphamide, and corticosteroids, and also Enbrel (ingredient name: etanercept), and Remicade (ingredient name: infliximab), Humira (ingredient name: adalimimab) of TNF-α antagonists as biological agents. Most of the therapeutic agents which have been so far developed for rheumatoid arthritis have adverse effects such as gastrointestinal disorders, and cannot significantly suppress the progress of joint damage.

TGF-β1, one of the cell therapeutic agent components of the present invention, is a multifunctional modulator for cell growth and differentiation. It has been reported that the neuropathic pain due to nerve injury is significantly reduced by intrathecal injection of recombinant TGF-β1 by targeting various types of cells with multi-differentiating potencies.

It has been recently reported that chondrocytes expressing TGF-β1 or fibroblasts are effective in treating degenerative arthritis (Lee K H et al., Hum Gene Ther 2001; 12: 1805-1813, SUN U. SONG et al. Tissue Engineering 2005; 11: 1516-1526), but the possibility of treating rheumatoid arthritis which has different causes and pathologies from those of degenerative arthritis has not been reported.

Accordingly, the present inventors have made efforts to develop a cell therapeutic agent having anti-inflammatory action and therapeutic effect for rheumatoid arthritis, and as a result, it has been confirmed that the symptoms of rheumatism are improved when a mixed cell composition of a chondrocyte and a cell expressing TGF-β1 is treated to a rheumatoid arthritis-induced animal model, whereby the present invention has been completed.

### SUMMARY

An aspect of the present invention is to provide a therapeutic use of (i) at least one of a chondrocyte and a cell capable of differentiating into a chondrocyte and (ii) a cell expressing TGF-β1, which is not a chemically synthesized drug, for treating an inflammatory disease induced by a hyperimmune response, wherein the cell in (ii) is derived from human embryonic kidney 293 (HEK-293) cell.

In order to accomplish the above aspect, an aspect of the present invention provides a composition for treating an inflammatory disease induced by a hyperimmune response, which includes (i) at least one of a chondrocyte and a cell capable of differentiating into a chondrocyte; and (ii) hChonJb#7 which is a cell into which a gene encoding TGF-β1 is introduced and is derived from human embryonic kidney 293 (HEK-293) cell.

Another aspect of the present invention provides a method of treating an inflammatory disease induced by a hyperimmune response, which includes administering to a subject with a composition including (i) a chondrocyte or a cell capable of differentiating into a chondrocyte; and (ii) a cell into which a gene encoding TGF-β1 is introduced, wherein the cell in (ii) is derived from human embryonic kidney 293 (HEK-293) cell.

Further, still another aspect of the present invention provides a use of a composition including (i) a chondrocyte or a cell capable of differentiating into a chondrocyte; and (ii) a cell into which a gene encoding TGF-β1 is introduced as a therapeutic agent for treating an inflammatory disease induced by a hyperimmune response, wherein the cell in (ii) is derived from human embryonic kidney 293 (HEK-293) cell.

Yet still another aspect of the present invention provides a method of inhibiting the expression or activity of a cytokine selected from the group consisting of IL-6, IL-17A, IL-1β, and TNF-α, which includes administering to a subject with a composition including (i) at least one of a chondrocyte and a cell capable of differentiating into a chondrocyte; and (ii) a cell into which a gene encoding TGF-β1 is introduced, wherein the cell in (ii) is derived from human embryonic kidney 293 (HEK-293) cell.

According to another embodiment, a composition includes at least one of a chondrocyte and a cell capable of differentiating into a chondrocyte, and a cell prepared by transducing a GP2-293 cell with a retroviral vector encoding TGF-β (transforming growth factor-beta).

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a diagram illustrating the mechanism of a therapeutic action of a mixed cell composition of the present invention for rheumatoid arthritis and degenerative arthritis;

FIG. 2 illustrates the results for the pain relief effect after treatment of a mixed cell to an animal model suffering with degenerative arthritis confirmed through the von Frey filament test;

FIG. 3 illustrates the results for H&E staining performed to confirm the cartilage status of the knee joint after treatment of a mixed cell to an animal model suffering with degenerative arthritis;

FIG. 4 illustrates the results for Masson's trichrome staining performed to confirm the cartilage status of the knee joint after treatment of a mixed cell to an animal model suffering with degenerative arthritis;

FIG. 5 illustrates the results for immunostaining performed to confirm the content of type 1 collagen and type 2 collagen in the cartilage after treatment of a mixed cell to an animal model suffering with degenerative arthritis;

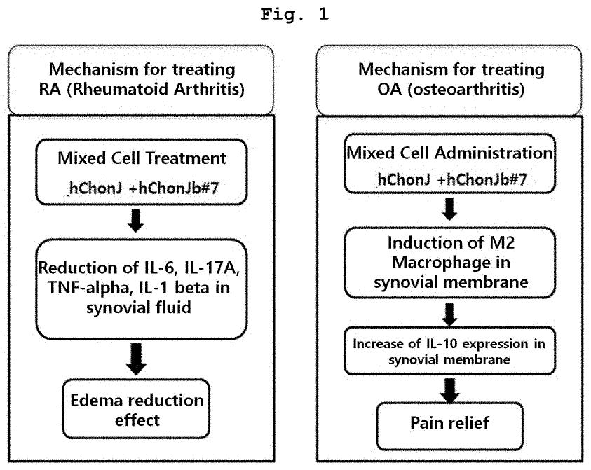

A and B of FIG. 6 illustrate the results for analysis on TGF-β1 and IL-10 expression levels in synovial lavage fluid in a degenerative arthritis-induced animal model;

A and B of FIG. 7 illustrate the results for immunochemical staining of the synovial membrane with an antibody to CD68 which is a surface antigen of a macrophage and IL-10 antibody to confirm the macrophage status of the cell-treated articular cavity of a degenerative arthritis-induced animal;

A of FIG. 8 illustrates the results for immunochemical staining of synovial membrane with antibodies to CD86 and arginase 1, B of FIG. 8 illustrates the image of A of FIG. 8 converted to an invert version by using Image-Pro Plus, and C of FIG. 8 is a graph illustrating CD86 and arginase 1 positive cells measurement;

FIG. 9 illustrates the results for qRT-PCR of the gene expression profile in the synovial membrane;

A and B of FIG. 10 are graphs illustrating the measurement results for edema reduction in the metatarsal region on the 19th day and 26th day after treatment of a mixed cell to a rheumatoid arthritis-induced animal model; and

FIG. 11 illustrates the results for a change in cytokine measured by harvesting the synovial lavage fluid from the articular cavity on the 28th day after treatment of a mixed cell to a rheumatoid arthritis-induced animal model.

### DETAILED DESCRIPTION

A mixed cell including an allogeneic (non-autologous) chondrocyte and a cell derived from human embryonic kidney 293 (HEK-293) cell expressing TGF-β1 is known as a cell-mediated gene therapeutic agent for degenerative arthritis. In a phase 2 clinical study for patients suffering with osteoarthritis, it has been confirmed that the mixed cell improves pain, activity and cartilage structures of patients suffering with osteoarthritis. However, a mechanism by which the mixed cell acts is not yet found.

The present inventors observed effects on pain and structural transformation after injection of the mixed cell to an osteoarthritis-induced animal model and confirmed whether the mixed cell induces an anti-inflammatory environment, and as a result, it has been confirmed that anti-inflammatory cytokines and M2 macrophages are increased in the arthritic knee joint treated with mixed cell to induce an anti-inflammatory environment that alleviates pain and induces joint regeneration.

Accordingly, it has been confirmed that the mixed cell induces an M2 macrophage-rich environment with immunosuppressive properties, and under the assumption that the mixed cell has an effect even on an inflammatory disease induced by a hyperimmune response, in a rheumatoid arthritis animal model, a chondrocyte and a cell derived from human embryonic kidney 293 (HEK-293) cell expressing TGF-β1 are mixed at a predetermined ratio and treated to knee joints. As a result, in a group treated with the mixed cell, it was confirmed that the metatarsal edema was reduced and further, it was confirmed that expression of IL-6, IL-17A, IL-1β, and TNF-α as cytokines that induced and promoted an inflammatory response in the synovial lavage fluid was reduced. A therapeutic effect by a single dose of the mixed cell lasted for 26 days.

Conventionally, materials such as methotrexate (MTX) used as a therapeutic agent for rheumatoid arthritis may cause digestive system and mouth ulcers and are at risk for causing hepatotoxicity, renal failure, and the like. Further, therapeutic agents such as anti-TNF-α antibodies including Remicade and the like are known to disturb a patient's innate immune system, resulting in the expression of tuberculosis (TB) from a tuberculosis carrier. In contrast, a cell therapeutic agent according to the present invention has an advantage in that the above adverse effects are not shown in a clinical test result for degenerative arthritis. Accordingly, the present inventors have confirmed the possibility that the mixed cell may be a novel therapeutic agent which overcomes disadvantages of conventional therapeutic agents for rheumatoid arthritis.

Therefore, an aspect of the present invention relates to a composition for treating an inflammatory disease induced by a hyperimmune response, which includes (i) at least one of a chondrocyte and a cell capable of differentiating into a chondrocyte, and (ii) a cell into which a gene encoding TGF-β1 is introduced, wherein the cell in (ii) is derived from human embryonic kidney 293 (HEK-293) cell.

Another aspect of the present invention relates to a method of treating an inflammatory disease induced by a hyperimmune response, which includes administering to a subject with a composition including (i) at least one of a chondrocyte and a cell capable of differentiating into a chondrocyte, and (ii) a cell into which a gene encoding TGF-β1 is introduced, wherein the cell in (ii) is derived from human embryonic kidney 293 (HEK-293) cell.

Still another aspect of the present invention relates to a use of a composition including (i) at least one of a chondrocyte and a cell capable of differentiating into a chondrocyte, and (ii) a cell into which a gene encoding TGF-β1 is introduced as a therapeutic agent for treating an inflammatory disease induced by a hyperimmune response, wherein the cell in (ii) is derived from human embryonic kidney 293 (HEK-293) cell.

In the present invention, the inflammatory disease induced by the hyperimmune response includes rheumatoid arthritis, multiple sclerosis, systemic lupus erythematosus, myasthenia gravis, colitis, type 1 diabetes, encephalitis, and the like, and among the diseases, it is particularly effective in the treatment for rheumatoid arthritis.

In the present invention, the cell capable of differentiating into the chondrocyte may be a cartilage precursor cell or a stem cell.

In the present invention, (i) the chondrocyte or the cell capable of differentiating into a chondrocyte and (ii) the cell into which a gene encoding TGF-β1 is introduced may be mixed at various mixing ratios, and the ratio of (i) and (ii) may be preferably 1:1 to 100:1 and more preferably 3:1, wherein the cell in (ii) is derived from human embryonic kidney 293 (HEK-293) cell.

In an aspect of the present invention, the composition for treating the inflammatory disease induced by the hyperimmune response includes a non-transduced human chondrocyte (hChonJ cell) and a cell (hereinafter also referred to as hChonJb#7) which is derived from human embryonic kidney 293 (HEK-293) cell and expresses TGF-β1, more specifically, a human allogeneic chondrocyte (hChonJ) and a cell expressing human TGF-β1. The two types of cells may be mixed at various mixing ratios. For example, these two cells may be mixed at a ratio of 3:1, that is, a ratio at which the hChonJ cell is 3 and the hChonJb#7 is 1. The mixed cell at the ratio of 3:1 and a production method thereof are disclosed in a thesis [Cytotherapy, 2012 February; 14 (2): 247-256] and U.S. Pat. Nos. 7,005,127 and 7,282,200.

In an aspect of the present invention, in an arthritis rat model treated with the mixed cell, the pain started to be reduced from 15 days after treatment and a pain relief effect was continued up to the time (an experiment end date) after 56 days after treatment. It has been confirmed that the pain relief was associated with a structural improvement of arthritic knee joints, and the treated mixed cell was found to be effective for both pain relief and cartilage regeneration. That is, the mixed cell used in the present invention is a cell-mediated gene therapy for pain relief and regeneration of cartilage tissue.

In another aspect of the present invention, with respect to the cartilage tissue in the arthritis rat model treated with the mixed cell, staining for type 1 collagen and type 2 collagen was performed and histologically analyzed, and as a result, it was confirmed that the mixed cell regenerated a hyaline cartilage tissue which was type 2 collagen-positive.

The macrophages which are innate immune cells are classified into activated “M1 macrophages” having a pro-inflammatory property and activated “M2 macrophages” having an immunosuppressive property according to an activation phenotype. IFN-γ is a key cytokine involved in the activation of the M1 macrophages and a TLR signal induced by pathogen recognition contributes to differentiation of the M1 macrophages. It has been known that the M1 macrophages increase expression of TNF-α, IL-12 and IL-23 and decrease expression of IL-10 to induce Th1 and Th17 immune responses which cause tissue damage. In contrast, the M2 macrophages increase the expression level of IL-10 and decrease expression of IL-12, thereby reducing inflammation and promoting tissue regeneration.

In an aspect of the present invention, TGF-β1 expressed in the hChonJb#7 which is a cell expressing TGF-β1 and IL-10 induced through the hChonJ which is a human chondrocyte induces generation of the M2 macrophages and it is determined that the generated M2 macrophages secrete IL-10 to create an anti-inflammatory environment in the joints treated with the mixed cell. The synovial membrane in the animal model treated with the mixed cell are histologically analyzed, and as a result, it can be confirmed that the M2 macrophages are increased (A to C of FIG. 8).

Further, it is determined that an immunosuppressive effect of the composition of the present invention is induced by an increase in expression of TGF-β1 and arginase 1, and in an aspect of the present invention, in the keen joint synovial membrane in an animal model of a group treated with the mixed cell, the expression of arginase 1 is increased.

It is known that the increase in the expression of arginase 1 and a decrease in L-arginine thereby cause a strong immunosuppressive effect in an immune system and further, it has been reported that the TGF-β1 induces transcription of arginase 1 and activity of arginase (Durante et al., Circulation, 103 (8), 1121-7, 2001). Accordingly, it is determined that the M2 macrophages may more efficiently regulate the immune in the present of TGF-β1.

Further, in the anti-inflammatory effect of the composition of the present invention, it is shown that IL-10 expressed in the M2 macrophage induced by treatment of the mixed cell activates STAT3 and PI3K and an anti-inflammatory effect is shown by Hmox-1 regulated thereby.

Meanwhile, recently, in the synovial membrane and the synovial fluid of a patient suffering with chronic rheumatoid arthritis, cytokines and growth factors, such as interleukin-1 (IL-1), interleukin-8 (IL-8), tumor necrosis factor-α (TNF-α), transforming growth factor β1 (TGF-β1), fibroblast growth factor (FGF), and platelet-derived growth factor (PDGF) have been detected (Nouri et al., Clin. Exp. Immunol. 55: 275-372, 1984; Thornton et al., Clin. Exp. Immunol. 86: 79-86, 1991; Saxne, et al., Arthritis Rheum. 31:1041-1045, 1988; Seitz et al., J. Clin. Invest. 87: 463-469, 1991; Lafyatis et al., J. Immunol. 143: 1142-1148, 1989; Melnyk et al., Arthrtis Rheum. 33: 493-500, 1990).

IL-6 is a cytokine known as B-cell stimulating factor 2 or interferon β2. IL-6 is a differentiation factor involved in activation of B lymphocytic cells (Hirano T. et al., Nature 324, 73-76, 1986) and is known as a multifunctional cytokine that affects an action of various types of cells (Akira, S. et al., Adv. In Immunology 54, 1-78, 1993).

It has been reported that in the serum or the synovial fluid of the patient suffering with the chronic rheumatoid arthritis, a large amount of interleukin-6 and soluble IL-6 receptors are present (Houssiau et al., Arthritis Rheum. 31: 784-788, 1988; Hirano et al., Eur. J. Immunol. 18: 1797-1801, 1988; Yoshioka et al., Japn. J. Rheumatol. In press). Even in the animal model of rheumatoid arthritis, a similar result is obtained (Takai et al., Arthritis Rheum. 32: 594-600, 1989; Leisten et al., Clin. Immunol. Immunopathol. 56: 108-115, 1990), and as a result, the IL-6 is known as a factor associated with the chronic rheumatoid arthritis.

Meanwhile, in a clinical development for rheumatoid arthritis, secukinumab as a biological agent is a high-affinity human monoclonal antibody that inhibits IL-17A activity. In the RA proof-of-concept (PoC) study, secukinumab was co-treated to an active RA patient treated with a stable dose of MTX. As a result, it was confirmed that a treatment group using secukinumab rapidly improved clinical symptoms of rheumatoid arthritis compared to placebo (Hueber et al. (2010) Sci. Transl. Med. 2(52):52-72). This is data showing that neutralization of IL-17A may be efficacious in the patient suffering with rheumatoid arthritis.

In another aspect of the present invention, it was confirmed that the mixed cell was treated to the paw of a rat in which rheumatoid arthritis was induced by treatment of a complete Freund's adjuvant (CFA) and the metatarsal edema was confirmed for 28 days. As a result, in the group treated with the mixed cell, it was confirmed that the metatarsal edema was decreased (A and B of FIG. 10), and as the result of analyzing IFN-γ, IL-6, IL-17A, IL-1β, and TNF-α which were Pro-inflammatory cytokine items that induced and promoted the inflammatory response, it was confirmed that in the mixed cell-treated group and the MTX-treated group, the metatarsal edema was statistically and significantly decreased as compared with a control.

Therefore, the composition of the present invention has an effect of inhibiting expression of IL-6, IL-17A, IL-1β, and TNF-α which are inflammatory cytokines that are excessively secreted from the joint synovial lavage fluid of the patient suffering with rheumatoid arthritis to reduce the edema.

Hereinafter, the present invention will be described in more detail through Examples. These Examples are just to exemplify the present invention, and it is apparent to expert in the field that it is interpreted that the scope of the present invention is not limited to these Examples.

### Example 1: Induction of Pain Relief and Regeneration of Col II Positive Cartilage by Treatment of Mixed Cell in Arthritis Animal Model

In the case of treating a mixed cell in a clinical phase 2 for patients suffering with arthritis, it was observed that pain was reduced and an environment of joints was structurally improved. In order to confirm a mechanism therefor, in a degenerative arthritis animal model induced by MIA, a mixed cell was treated and a joint histological examination and a von Frey filament test were performed.

1-1: Preparation of MIA-Induced Arthritis Animal Model

In preparation of an animal model, a 6-week-old male rat (Spargue-Dawley, 200 to 225 g, Nara Biotech, Korea) were used. An animal experiment was conducted under a veterinary control after approval of the Kolon Life Science (Korea) Institutional Animal Care and Use Committee (IACUC No. KLS IACUC 2013-04).

In order to induce arthritis, 50 μl of an MIA (monosodium iodoacetate, Sigma, USA) solution having a concentration of 60 mg/mL was treated to the articular cavity of the left knee of a rat by using a 31G syringe.

1-2: Preparation of Cells for Cell Therapy

The hChonJ used in the present invention is a human-derived chondrocyte and the hChonJb#7 is a GP2-293 cell transformed to secrete TGF-β. GP2-293 cell is a human-derived cell line that originated from human embryonic kidney 293 (HEK-293) cell. Preparation methods and mixing ratios of the cells are disclosed in a thesis [Cytotherapy, 2012 February; 14(2): 247-256] and U.S. Pat. Nos. 7,005,127 and 7,282,200.

The hChonJb#7 may be prepared by injecting a cDNA of TGF-β1 into cells according to a known method. For example, a promoter such as methallothionein and a cDNA of TGF-β1 are included in a known vector [for example, pUC19 (including an ampicillin resistance gene) by Gibco-BRL Corporation] having a resistance gene such as ampicillin and neomycin to prepare a vector including the cDNA of TGF-β. For example, a retroviral vector encoding TGF-β may be produced by co-transfection of the packaging cell line with two plasmids which is pKEB1 and pKVSV-G. Thereafter, the retroviral vector is infected into a GP2-293 cell to transduce GP2-293 cell. The transduced GP2-293 cells were irradiated during preparation to prevent replication, thereby preparing the hChonJb#7.

1-3: Confirmation of Pain Relief and Cartilage Regeneration Effects by Treatment of Mixed Cell

After 2 weeks of MIA injection, mixed cells {(1.2×10⁶ cells: hChonJ (9×10⁵ cells)+hChonJb#7 (3.0×10⁵ cells)}, hChonJ (9×10⁵ cells), hChonJb#7 (3.0×10⁵ cells) or a control {CryoStor-10 (CS-10)} were treated to the articular cavity of the left knee. General symptoms were observed, and body weights were measured once a week.

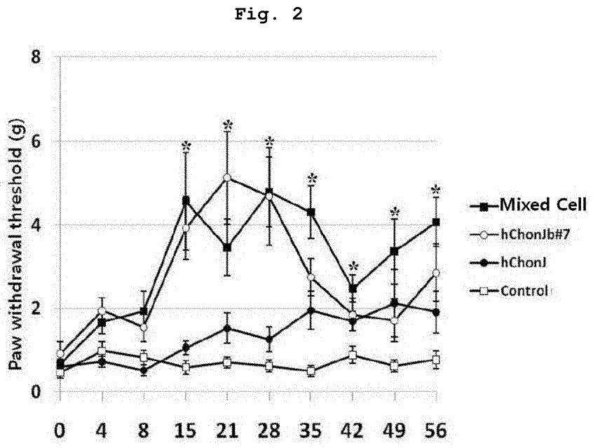

The pain relief was observed by performing a von Frey filament test, and as a result, as illustrated in FIG. 2, pain levels decreased from 15 days after cell treatment, and such effects lasted until the end of observation (56th day after treatment). In the case of a group treated with hChonJb#7 (3.0×10⁵ cells) expressing TGF-β, the pain relief was observed from 15 days, but after 6 weeks, it was confirmed that the effect disappeared. In contrast, in a group treated with hChonJ (9×10⁵ cells) which was a normal human chondrocyte, the pain relief effect was not observed.

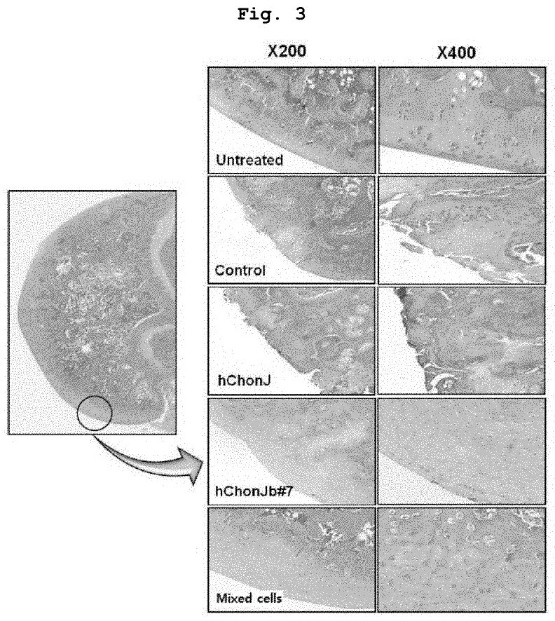

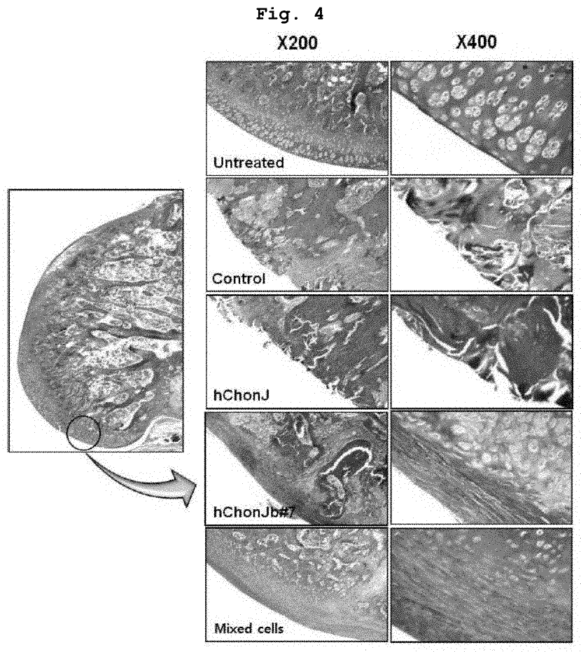

In order to confirm whether the pain relief effect of the mixed cell is associated with cartilage regeneration, left knee joints in 5 experimental groups were harvested and a histological analysis was performed through H&E staining and Masson's trichrome staining (see FIGS. 3 and 4).

In a control (CS-10) and an hChonJ-treated group, cartilage regeneration was not confirmed. In contrast, in an hChonJb#7-treated group and a mixed cell-treated group, more cartilage was observed than other controls.

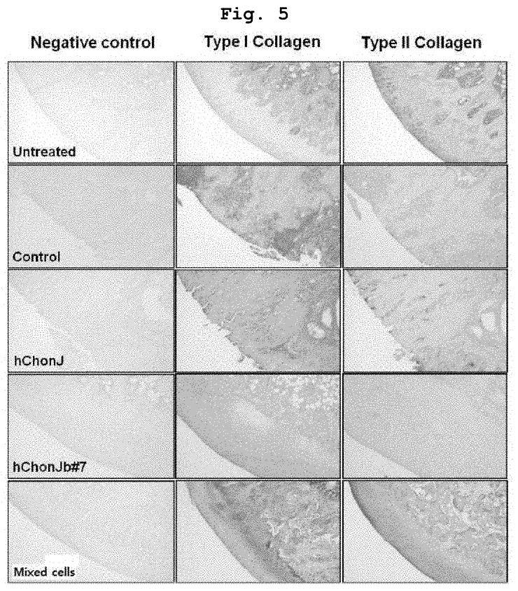

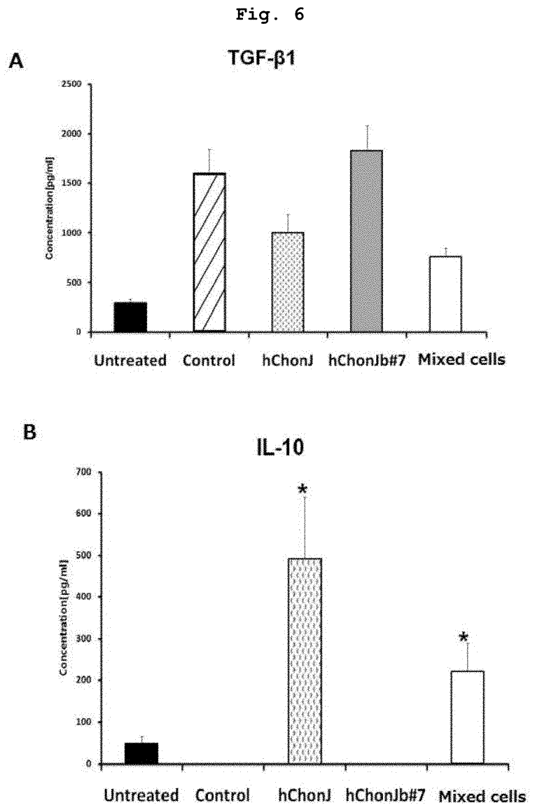

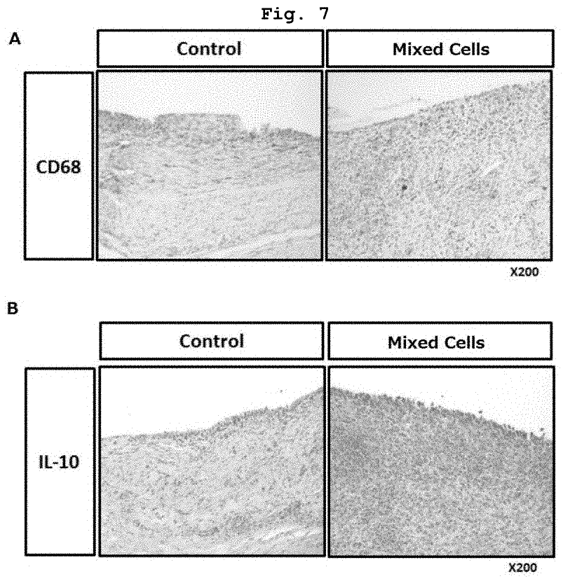

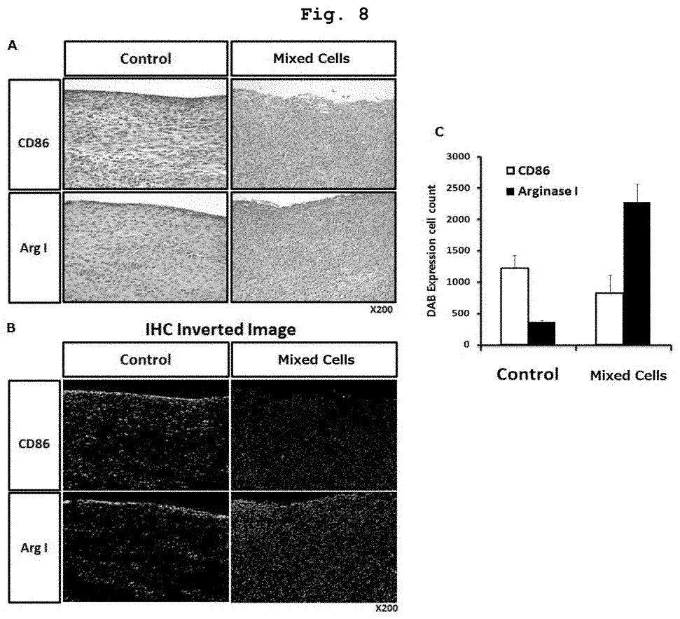

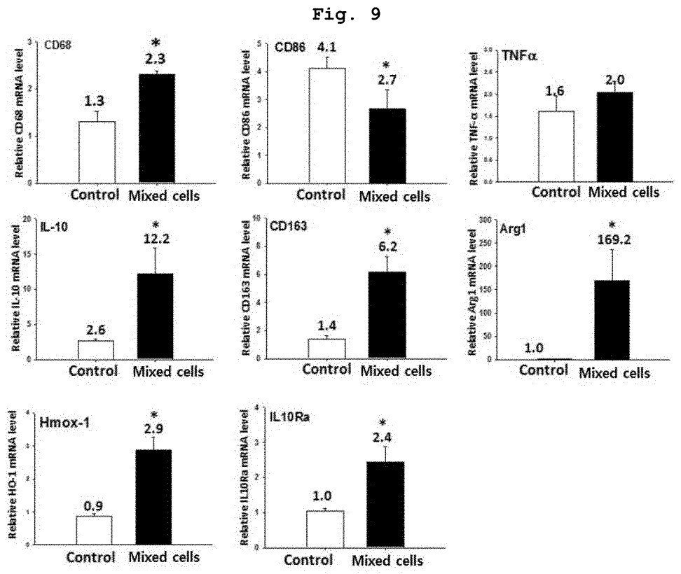

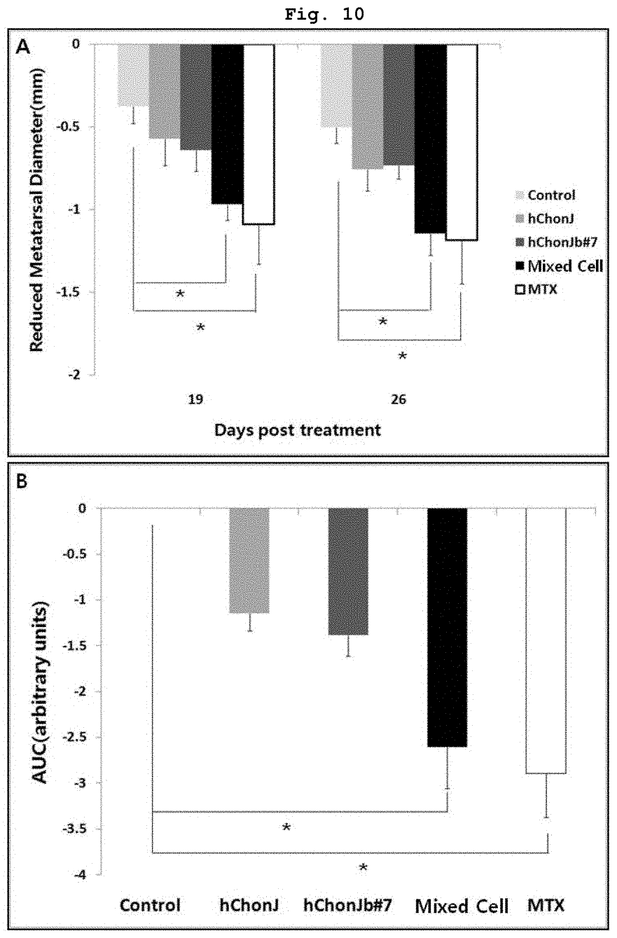

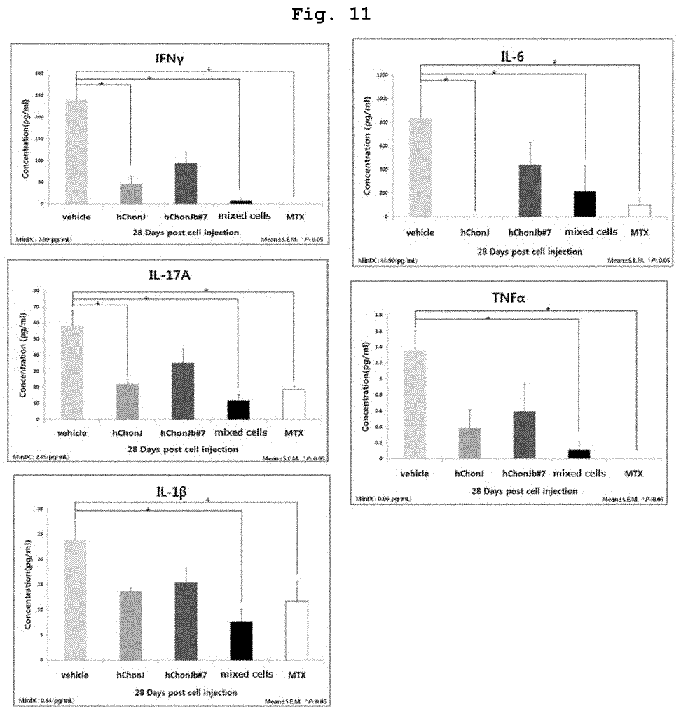

Further, as the immunohistochemical staining result, as illustrated in FIG. 5, it was confirmed that the content of type II collagen in the cartilage in the mixed cell-treated group was higher than that in the hChonJb#7-treated group, and from the result, it can be seen that the cartilage in the mixed cell-treated group is close to the hyaline cartilage.

In order to evaluate the improvement degree of the joint status, as a method disclosed in the thesis [Kobayasi, K., J. et al., J. Vet. Med. Sci., 65:1195-1199, 2003], the histopathological results and the pathology scores of the H&E-stained joint tissue were evaluated and the results were summarized in the following Table 2. In the experimental results below, the cartilage in the mixed cell-treated group was most improved.

TABLE 2

| | | Treatment No. of Animals | | | | |
|---|---|---|---|---|---|---|
| | | Untreated | Control | hChonJ | hChonJb#7 | Invossa |
| Structural change in the joint | | 5 (x3) | 6 (x3) | 6 (x3) | 6 (x3) | 6 (x3) |
| Surface irregularities | 1 | 2/15 | 3/18 | 0/18 | 5/18 | 7/18 |
| | 2 | 0/15 | 4/18 | 1/18 | 2/18 | 4/18 |
| | 3 | 0/15 | 10/18 | 14/18 | 6/18 | 4/18 |
| Average pathology score | | 0.13 | 2.28 | 2.44 | 1.50 | 1.50 |
| Standard Error | | 0.09 | 0.23 | 0.22 | 0.29 | 0.25 |
| Ulceration | 1 | 1/15 | 3/18 | 3/18 | 8/18 | 7/18 |
| | 2 | 0/15 | 8/18 | 7/18 | 6/18 | 6/18 |
| | 3 | 0/15 | 7/18 | 7/18 | 3/18 | 0/18 |
| Average pathology score | | 0.07 | 2.22 | 2.11 | 1.61 | 1.06 |
| Standard Error | | 0.07 | 0.18 | 0.18 | 0.21 | 0.19 |
| Fibrillation of | 1 | 0/15 | 3/18 | 3/18 | 1/18 | 3/18 |
| cartilage surface | 2 | 0/15 | 2/18 | 6/18 | 6/18 | 5/18 |
| | 3 | 0/15 | 7/18 | 5/18 | 6/18 | 1/18 |
| Average pathology score | | 0 | 1.56 | 1.67 | 1.72 | 0.89 |
| Standard Error | | 0 | 0.32 | 0.26 | 0.29 | 0.25 |
| Disorganization | 1 | 1/15 | 1/18 | 7/18 | 9/18 | 6/18 |
| of chondrocytes | 2 | 0/15 | 10/18 | 5/18 | 8/18 | 7/18 |
| | 3 | 0/15 | 5/18 | 4/18 | 0/18 | 0/18 |
| Average pathology score | | 0.07 | 2.00 | 1.61 | 1.39 | 1.11 |
| Standard Error | | 0.07 | 0.23 | 0.24 | 0.15 | 0.20 |
| Exposure of | 1 | 0/15 | 7/18 | 6/18 | 4/18 | 6/18 |
| subchondral bone | 2 | 0/15 | 1/18 | 0/18 | 4/18 | 1/18 |
| | 3 | 0/15 | 8/18 | 8/18 | 0/18 | 0/18 |
| Average pathology score | | 0 | 1.83 | 1.67 | 0.67 | 0.44 |
| Standard Error | | 0 | 0.29 | 0.30 | 0.21 | 0.15 |
| Cellular changes | 1 | 2/15 | 7/18 | 6/18 | 9/18 | 9/18 |
| of chondrocyte | 2 | 0/15 | 2/18 | 3/18 | 4/18 | 1/18 |
| H & E staining | 3 | 0/15 | 4/18 | 3/18 | 1/18 | 2/18 |
| Average pathology score | | 0.13 | 1.28 | 1.17 | 1.11 | 0.94 |
| Standard Error | | 0.09 | 0.27 | 0.26 | 0.17 | 0.20 |
| Degeneration/Necrosis | 1 | 0/15 | 3/18 | 2/18 | 5/18 | 6/18 |
| | 2 | 0/15 | 4/18 | 8/18 | 5/18 | 4/18 |
| | 3 | 0/15 | 11/18 | 8/18 | 5/18 | 0/18 |
| Average pathology score | | 0 | 2.44 | 2.33 | 1.67 | 0.78 |
| Standard Error | | 0 | 0.19 | 0.17 | 0.26 | 0.20 |
| H & E staining | 1 | 0/15 | 5/18 | 4/18 | 10/18 | 10/18 |
| Reduction of | 2 | 0/15 | 5/18 | 5/18 | 5/18 | 2/18 |
| staining in cartilage | 3 | 0/15 | 6/18 | 7/18 | 1/18 | 0/18 |
| Average pathology score | | 0 | 1.83 | 1.94 | 1.28 | 0.78 |
| Standard Error | | 0 | 0.23 | 0.23 | 0.17 | 0.14 |
| Total pathology score | | 0.4 ± 0.32 | 15.44 ± 1.94 | 14.94 ± 1.88 | 10.94 ± 1.75 | 7.5 ± 1.57 |

0: Normal
1: Slight,
2: Moderate,
3: Severe

### Example 2: Analysis of Effect on Secretion of Anti-Inflammatory Cytokine by Treatment of Mixed Cell

An effect on a cytokine expression profile of the synovial fluid in the articular cavity by treatment of mixed cells in an animal model was confirmed.

In order to confirm whether macrophages are induced at a cell site, it was confirmed that CD68 positive cells were present in the synovial membrane.

The paraffin-embedded synovial membrane was microsected with a thickness of 4 μm to prepare a tissue slide, and then immunohistochemical staining was performed by using CD68 antibodies (Novus, Co, USA).

As a result, as illustrated in A of FIG. 7, CD68-positive cells were significantly increased in the mixed cell-treated group.

IL-10 was known to exhibit the pain relief effect. An arthritis model was created based the above report and the mixed cell was treated, and then expression of IL-10 was confirmed. The expression of IL-10 was measured in the synovial lavage fluid of the arthritis-induced rat by using a cytokine magnetic bead panel 96-well plate assay (#RECYTMAG-65K, Milipore, USA).

As a result, as illustrated in B of FIG. 6, in the mixed cell-treated group and the hChonJ-treated group, it was confirmed that the expression of IL-10 was increased. From the histological analysis result, it was confirmed that IL-10-positive cells were increased in the mixed cell-treated group. These results indicate that the treatment of the mixed cell in the arthritic knee joints induces IL-10 which is an anti-inflammatory cytokine.

### Example 3: Confirmation of Induction of M2 Type Macrophage-Rich Environment by Mixed Cell

There are two different types of macrophages. The M1 type macrophages are responsible for acute inflammation to secrete high levels of inflammatory cytokines such as IL-12 and TNF-α, and the M2 macrophages play an important role in wound healing and anti-inflammatory regulatory functions.

In Example 2, the high level of IL-10 expression was confirmed in the mixed cell-treated group, and as a result, in order to confirm whether the mixed cell induces the macrophages in the knee of the arthritis-induced animal, in the synovial membrane of the animal model, the expression of CD86 as a marker of the M1 macrophage and the expression of arginase 1 as a marker of the M2 macrophage were confirmed.

To this end, the paraffin-embedded synovial membrane was immuno-stained by using a CD86 antibody (AbD serotec, NC, USA) and a mouse arginase 1 antibody (BD Bioscience, USA).

As a result, as illustrated in A to C of FIG. 8, the expression of CD86 had no difference in the control and the mixed cell-treated group, but arginase 1 was significantly high in the mixed cell-treated group. It is shown that the mixed cells induce the M2 macrophages.

### Example 4: Confirmation of Induction of M2 Macrophage-Rich Environment by Mixed Cell: RNA

In order to confirm the induction of the M2 macrophages confirmed in Example 3 in the gene expression level, the total RNA was isolated from the synovial membrane of the animals and the expression of genes related with the M1 and M2 macrophages was confirmed.

The RNA in the synovial membrane was isolated by using a RNeasy Lipid Tissue Mini kit (QIAGEN, USA) and cDNA was synthesized from the isolated RNA by using a SuperScript™ III First-Strand Synthesis System (Invitrogen, USA).

Thereafter, real-time PCR was performed by using ABI7900 equipment. A PCR reaction mixture used 1 μL cDNA, 0.2 μM of each primer, and 10 μL SYBR Premix Ex Taq (TAKARA, Japan) at a total of 50 μL, and a reaction condition was 10 seconds at 95° C. and 30 seconds at 60° C. and a total of 40 cycles were performed. A gene expression level was confirmed by comparing an expression level of a β-actin gene by using a 2-ΔΔCt method and the used qRT-PCR primers were listed in Table 3.

TABLE 3
Primer sequences used for qRT-PCR

| Gene | Forward primer | Reverse primer |
|---|---|---|
| CD80 | TGCTGGTTGGTCTT | TGACTGCTCTTCA |
| | TTCCA | GAACAAAA |
| | (SEQ ID NO. 1) | (SEQ ID NO. 2) |
| CD86 | TCCTCCAGCAGTGG | TTTGTAGGTTTCG |
| | GAAACA | GGTATCCTTGC |
| | (SEQ ID NO. 3) | (SEQ ID NO. 4) |
| CD163 | CTCAGCGTCTCTGC | GGCCAGTCTCAGT |
| | TGTCAC | TCCTTCTT |
| | (SEQ ID NO. 5) | (SEQ ID NO. 6) |
| Arg1 | TTGATGTTGATGGA | TCTCTGGCTTATG |
| | CTGGAC | ATTACCTTC |
| | (SEQ ID NO. 7) | (SEQ ID NO. 8) |
| IL-1β | TCCAGGATGAGGAC | TCGTCATCATCCC |
| | CCAAGC | ACGAGTCA |
| | (SEQ ID NO. 9) | (SEQ ID NO. 10) |
| TNFα | ACTGAACTTCGGGG | GCTTGGTGGTTTG |
| | TGATTG | CTACGAC |
| | (SEQ ID NO. 11) | (SEQ ID NO. 12) |
| IL-10 | CAAGGCAGTGGAGC | CCGGGTGGTTCAA |
| | AGGTGA | TTTTTCATT |
| | (SEQ ID NO. 13) | (SEQ ID NO. 14) |
| Hmox-1 | AGAGTTTCCGCCTC | CGGGACTGGGCTA |
| | CAACCA | GTTCAGG |
| | (SEQ ID NO. 15) | (SEQ ID NO. 16) |
| IL10Ra | CTGGTCACCCTGCC | AGGCATGGCCAAA |
| | ATTGAT | ATACAAAGAAAC |
| | (SEQ ID NO. 17) | (SEQ ID NO. 18) |
| CD68 | ACTGGGGCTCTTGG | CCTTGGTTTTGTT |
| | AAACTACAC | CGGGTTCA |
| | (SEQ ID NO. 19) | (SEQ ID NO. 20) |
| β-actin | AGTTCGCCATGGAT | AAGCCGGCCTTGC |
| | GACGAT | ACAT |
| | (SEQ ID NO. 21) | (SEQ ID NO. 22) |

Primer sequences for target genes for qRT-PCR of rat genes. CD = Cluster of Differentiation, Arg = Arginase, IL = Interleukin, TNFα = Tumor necrosis factors alpha, Hmox = Heme oxygenase

A macrophage related gene expression profile is illustrated in FIG. 9. The expression of CD68 was high in the mixed cell-treated group. TNF-α had no difference between the control and the mixed cell-treated group. IL-10 as an anti-inflammatory cytokine was very highly expressed in the mixed cell-treated group.

In order to confirm whether an increase in mRNA expression of IL-10 causes protein expression, a multiple cytokine assay in the synovial membrane was performed. As a result, as illustrated in A and B of FIG. 10, it was confirmed that in the mixed cell-treated group, a level of the IL-10 protein was rapidly increased. It was confirmed that the mRNA levels of IL-10RA (interleukin 10 receptor alpha subunit) and Hmox1 (heme oxygenase 1) which were additional macrophage markers were significantly increased.

When summarizing the results, in the case of administering the mixed cell in the arthritis model, it can be seen that induction of the M2 macrophages is increased.

### Example 5: Confirmation of Efficacy of Mixed Cell in Rheumatoid Arthritis Animal Model

5-1: Preparation of Rheumatoid Arthritis Animal Model

In a preparation of a rheumatoid arthritis-induced animal model, a 7-week-old female rat (160 to 180 g Lewis, Central laboratory animals, Japan) was used. The animal experiment was conducted after approval of the Kolon Life Science (Korea) Institutional Animal Care and Use Committee (IACUC No. KLS IACUC 2015-07).

In order to induce rheumatoid arthritis, 50 μl of a CFA (Complete Freud's Adjuvant, manufacturer: Chondrex, Inc.) solution having a concentration of 5 mg/mL was treated to the subcutaneous sole of a rat by using a 31G syringe. After 3 weeks, induction of arthritis was confirmed by measuring the size of the metatarsal edema using calipers, and thereafter, when the edema was most severely induced, a test material was treated to the Lewis-rat left knee articular cavity.

5-2: Treatment of Test Material to Animal Model

Rheumatoid arthritis-induced rats were set to 6 rats per group, and after 3 weeks of CFA treatment, mixed cells {(1.2×10⁶ cells: hChonJ (9×10⁵ cells)+hChonJb#7 (3.0×10⁵ cells)}, hChonJ (9×10⁵ cells), hChonJb#7 (3.0×10⁵ cells), and a negative control (CS-10) were treated to the left knee articular cavity. In the case of a positive control, 0.4 mg/kg of MTX (Methotrexate) was orally treated once a week. The metatarsal diameter and the body weight were measured once per three days.

In Table 4, a treatment condition for each experimental group was illustrated.

TABLE 4
treatment condition for each experimental group

| | | | Number of | Dosing | Method of |
|---|---|---|---|---|---|
| experimental group | | Cell/50 μl* | Animal | Schedule | Treatment |
| AIA | Negative | CS-10 | 6 | Single after | administration |
| | Control Group | | | 3 weeks | to the |
| | hChonJ | 9 × 10⁵ | 6 | inducing AIA | articular cavity |
| | hChonJb#7 | 3 × 10⁵ | 6 | | |
| | Mixed cell group | 1.2 × 10⁶ | 6 | | |
| | MTX | 0.4 mg/kg | 6 | Once a week | Oral |
| | | | | (21 d) | treatment |

Treatment Condition for Each Experimental Group

5-3: Effect of Mixed Cell in Rheumatoid Arthritis Animal Model

The test material was treated to the arthritis model and the size of the metatarsal edema was measured by a caliper, and the result was illustrated in A and B of FIG. 10. After treatment of the control (CS-10), hChonJ, hChonJb#7, mixed cell, and MTX (Methotrexate), the decrease in the size of the edema was measured on the 19th day and the 26th day, and as a result, in the mixed cell-treated group and the MTX-treated group, the decrease was statistically and significantly shown as compared with the control.

5-4: Analysis of Cytokine Secretion Profile by Treatment of Mixed Cell

A sample was treated to the arthritis model, the synovial lavage fluid was harvested from the left knee articular cavity of the experimental animal rat on the 28th day, and the cytokines were analyzed, and the result was illustrated in FIG. 11. The synovial lavage fluid was collected from the rat articular cavity by administering 50 μL of a saline solution for injection by a 31G insulin syringe and then immediately collecting the solution while the syringe pierced. As the cytokines, IFN-γ, IL-6, IL-17, IL-1β and TNF-α were analyzed and the analysis was performed by using Rat Cytokine/Chemokine multiplex (RECYTMAG-65K, Millipore). IFN-γ, IL-6, IL-17, IL-1β and TNF-α, which were Pro-inflammatory cytokine items inducing and promoting an inflammatory response, were analyzed and as a result, in the mixed cell-treated group and the MTX (Methotrexate)-treated group, the cytokines were statistically and significantly decreased as compared with the control.

The mixed cell composition of the present invention can improve symptoms of inflammatory diseases including rheumatoid arthritis and suppress a hyperimmune response by cell therapy without adverse effects from the other types of conventional drug therapy.

Although the specific part of the present invention has been described in detail, it is obvious to those expert in the field that such a specific description is just a preferred embodiment and the scope of the present invention is not limited. Thus, the substantial scope of the present invention will be defined by the appended claims and equivalents thereof.

A sequence listing electronically submitted with the present application on Dec. 7, 2021 as an ASCII text file named 20211207_Q43220LC36T-CIP_TU_SEQ, created on Dec. 7, 2021 and having a size of 5,000 bytes, is incorporated herein by reference in its entirety.

What is claimed is:

1. A composition comprising:
(i) at least one of a chondrocyte and a cell capable of differentiating into a chondrocyte; and
(ii) hChonJb#7 which is a cell into which a gene encoding TGF-β (transforming growth factor-beta) is introduced and is derived from human embryonic kidney 293 (HEK-293) cell.

2. The composition of claim 1, wherein the cell capable of differentiating into a chondrocyte is a cartilage precursor cell or a stem cell.

3. The composition of claim 1, wherein a ratio of said (i) to said (ii) ranges from 1:1 to 100:1.

4. The composition of claim 1, wherein the ratio of said (i) to said (ii) ranges from 1:1 to 3:1.

5. The composition of claim 1, wherein the composition comprises:
(i) the chondrocyte; and
(ii) the cell into which the gene encoding TGF-β is introduced.

6. The composition of claim 1, wherein the composition comprises:
(i) the cell capable of differentiating into the chondrocyte; and
(ii) the cell into which the gene encoding TGF-β is introduced.

7. A method of treating an inflammatory disease induced by a hyperimmune response, the method comprising:
administering the composition of claim 1 to a subject.

8. The method of claim 7, wherein the administering comprising administering the composition of claim 1 directly to an articular cavity of the subject.

9. A method of inhibiting the expression or activity of a cytokine selected from the group consisting of IL-6, IL-17A, IL-1β, and TNF-α, the method comprising:
administering the composition of claim 1 to a subject.

10. The method of claim 9, wherein the administering comprising administering the composition of claim 1 directly to an articular cavity of the subject.

11. The method of claim 9, wherein the inflammatory disease induced by the hyperimmune response is selected from the group consisting of rheumatoid arthritis, multiple sclerosis, systemic lupus erythematosus, myasthenia gravis, colitis, type 1 diabetes, and encephalitis.

12. The method of claim 11, wherein the inflammatory disease induced by the hyperimmune response is rheumatoid arthritis.

13. A composition comprising:
at least one of a chondrocyte and a cell capable of differentiating into a chondrocyte; and
a cell prepared by transducing a GP2-293 cell with a retroviral vector encoding TGF-β (transforming growth factor-beta) and irradiating the transduced GP2-293 cell, wherein the retroviral vector encoding TGF-β is produced by co-transfection of a packaging cell line with two plasmids of pKEB1 and pKVSV-G.

14. The composition of claim 13, wherein the at least one of the chondrocyte and the cell capable of differentiating into the chondrocyte includes a human allogeneic chondrocyte.

15. The composition of claim 13, wherein a ratio of the human allogenic chondrocyte to the cell prepared by transducing GP2-293 cell ranges from 1:1 to 3:1.

16. A method of treating rheumatoid arthritis, the method comprising:
administering to a subject a composition comprising (i) a chondrocyte; and (ii) a cell into which a gene encoding TGF-β (transforming growth factor-beta) is introduced, wherein the cell in (ii) is hChonJb#7 which is derived from human embryonic kidney 293 (HEK-293) cell,
wherein the administration of the composition has an effect of inhibiting expression of IL-6, IL-17A, IL-1β, and TNF-α in a synovial lavage fluid in the articular cavity of the subject.

17. The method of claim 16, wherein a ratio of said (i) to said (ii) ranges from 1:1 to 100:1.

18. The method of claim 16, wherein the ratio of said (i) to said (ii) ranges from 1:1 to 3:1.

19. The method of claim 16, wherein the administering comprising administering directly to an articular cavity of the subject.

20. The method of claim 16, wherein the administering comprising administering directly to an articular cavity of the subject via an intra-articular injection.


SEQUENCE LISTING (22 sequences)

SEQ ID NO: 1 - DNA, 19 bp, Artificial Sequence
  primer
tgctggttggtcttttcca

SEQ ID NO: 2 - DNA, 21 bp, Artificial Sequence
  primer
tgactgctcttcagaacaaaa

SEQ ID NO: 3 - DNA, 20 bp, Artificial Sequence
  primer
tcctccagcagtgggaaaca

SEQ ID NO: 4 - DNA, 24 bp, Artificial Sequence
  primer
tttgtaggtttcgggtatccttgc

SEQ ID NO: 5 - DNA, 20 bp, Artificial Sequence
  primer
ctcagcgtctctgctgtcac

SEQ ID NO: 6 - DNA, 21 bp, Artificial Sequence
  primer
ggccagtctcagttccttctt

SEQ ID NO: 7 - DNA, 20 bp, Artificial Sequence
  primer
ttgatgttgatggactggac

SEQ ID NO: 8 - DNA, 22 bp, Artificial Sequence
  primer
tctctggcttatgattaccttc

SEQ ID NO: 9 - DNA, 20 bp, Artificial Sequence
  primer
tccaggatgaggacccaagc

SEQ ID NO: 10 - DNA, 21 bp, Artificial Sequence
  primer
tcgtcatcatcccacgagtca

SEQ ID NO: 11 - DNA, 20 bp, Artificial Sequence
  primer
actgaacttcggggtgattg

SEQ ID NO: 12 - DNA, 20 bp, Artificial Sequence
  primer
gcttggtggtttgctacgac

SEQ ID NO: 13 - DNA, 20 bp, Artificial Sequence
  primer
caaggcagtggagcaggtga

SEQ ID NO: 14 - DNA, 22 bp, Artificial Sequence
  primer
ccgggtggttcaatttttcatt

SEQ ID NO: 15 - DNA, 20 bp, Artificial Sequence
  primer
agagtttccgcctccaacca

SEQ ID NO: 16 - DNA, 20 bp, Artificial Sequence
  primer
cgggactgggctagttcagg

SEQ ID NO: 17 - DNA, 20 bp, Artificial Sequence
  primer
ctggtcaccctgccattgat

SEQ ID NO: 18 - DNA, 25 bp, Artificial Sequence
  primer
aggcatggccaaaatacaaagaaac

SEQ ID NO: 19 - DNA, 23 bp, Artificial Sequence
  primer
actggggctcttggaaactacac

SEQ ID NO: 20 - DNA, 21 bp, Artificial Sequence
  primer
ccttggttttgttcgggttca

SEQ ID NO: 21 - DNA, 20 bp, Artificial Sequence
  primer
agttcgccatggatgacgat

SEQ ID NO: 22 - DNA, 17 bp, Artificial Sequence
  primer
aagccggccttgcacat